(19) United States
(12) Patent Application Publication
(10) Pub. No.: US 2003/0120201 A1
(43) Pub. Date: Jun. 26, 2003

(54) Methods and devices for sclerotherapy

(76) Inventor: R. Patrick Abergel, Santa Monica, CA (US)

Correspondence Address: PERKINS COIE LLP, POST OFFICE BOX 1208, SEATTLE, WA, 98111-1208, US

(21) Appl. No.: 10/029,321

(22) Filed: Dec. 21, 2001

(51) Int. Cl. A61M 1/00

(52) U.S. Cl. 604/28; 604/191

(57) ABSTRACT

In a method for sclerotherapy for treating varicose veins, a flushing solution, such as sterile saline solution, is initially injected into the vein or vessel being treated. The flushing solution displaces or flushes out blood from the treatment site of the vessel. A sclerosing agent is then injected into the treatment site. The displacement of blood before introduction of the sclerosing agent reduces complications. A syringe assembly useful for performing the method has first and second reservoirs sealed off from each by an end cap. The end cap is removed just before use. A needle is attached and is connected to both reservoirs. Flushing solution is delivered from the first reservoir followed by sclerosing solution delivered from the second reservoir, without removing the needle from the vessel.

### FIELD OF THE INVENTION

The field of the invention is sclerotherapy. More specifically, the invention relates to the treatment of spider and varicose veins via sclerotherapy. The invention further relates to a novel syringe assembly useful in sclerotherapy, as well as in other medical applications.

### BACKGROUND OF THE INVENTION

Spider veins or telangiectasias of the legs are common conditions, especially among women. With this condition, small dark-colored veins form on the legs, just underneath the skin surface. These types of veins can form anywhere on the legs between the thigh and ankle. They often have a web or sunburst pattern, but may also be formed as short, somewhat random line segments. In many cases, spider veins are largely unnoticeable, when localized in a small area. However, when larger areas of skin are affected, spider veins can be perceived as having a detrimental appearance on the skin.

Varicose veins are larger veins, in comparison to spider veins. Varicose veins may protrude or be raised above the skin surface. They typically have a blue or purple color. A varicose vein generally contains stagnant or refluxing blood, which is out of circulation. Consequently, a varicose vein no longer functions to channel blood flow back to the circulatory system or the heart. Larger veins have valves which maintain blood flow in the forward direction. If the valves fail, blood accumulates under pressure, causing the veins of the leg to engorge. These varicose veins often appear as bulging, and have a rope-like or thread-like appearance. In more severe cases, these vascular disorders can result in aching, throbbing, swelling, or other conditions requiring medical treatment. Moreover, many patients having varicose veins, even without these symptoms, become distressed by the appearance of the varicose veins. Consequently, various treatments have been developed for both medical and cosmetic reasons. These treatments include surgery for severe cases, as well as sclerotherapy, typically used for smaller varicose veins closer to the skin surface. In the past, sclerotherapy has been performed by injecting a sclerosing agent into the vein. This non-surgical procedure destroys the varicose vein by irritating the vein wall, and causing the vein to close up. Procedures using ultrasound, or an electrosurgical electrode in combination with sclerotherapy have also been proposed. In general, sclerotherapy is a proven, safe, and effective technique.

Notwithstanding the successes of sclerotherapy, complications can occur with these treatments. One such complication is ulceration. This complication results when a sclerosing solution is inadvertently injected intra-dermally or into surrounding tissue, rather than into the intended injection site in a vein. The sclerosing solution delivered outside of the vein can cause ulceration of the skin and surrounding tissue. As it may be difficult to consistently position the needle into the vein, this type of ulceration is a common complication. Hyperpigmentation is another complication. This complication results due to leakage of a blood component, hemosiderin pigment, from the damaged blood vessel or vein. Another complication is mat-like telangiectasia, which is the appearance of new, small blood vessels. Mat-like telangiectasia is believed to result from injection of an excessive amount of sclerosing solution.

Accordingly, it is an object of the invention to provide improved methods and devices for treating spider and varicose veins via sclerotherapy.

In certain medical procedures, it is advantageous to be able to inject different solutions into a single injection site, either simultaneously, or sequentially. Currently, this generally may require two separate injections. As a result, the two injections may not be located at the same location. In addition, two separate injections requires more time to provide, consumes more syringes and needles (generating more medical waste), and causes more pain and risk of infection to the patient.

Accordingly, it is also an object of the invention to provide a novel syringe assembly useful for injecting two separate solutions in a single procedure.

### SUMMARY OF THE INVENTION

To these ends, in a first aspect, a method for treating a spider vein or a varicose vein via sclerotherapy includes injecting a flushing solution into the vein, to flush out blood from the section of the vein treated. A sclerosing solution is then injected into the blood vessel. The flushing solution and sclerosing solution are preferably injected from a single hypodermic needle. This allows both the flushing and sclerosing solutions to be injected sequentially at the same location and via a single injection or piercing of the skin and vein.

In a second aspect of the invention, a syringe assembly for providing sclerotherapy has two separate reservoirs, chambers, or syringes. The first reservoir contains a flushing solution, preferably sterile saline solution. The second reservoir contains a sclerosing agent. A needle is attached at one end of the syringe assembly. Both reservoirs are connectable into the preferably 30-gauge needle. Each reservoir has a separate plunger.

In use, in a third aspect of the invention, the needle is positioned in the vein or vessel to be treated. The plunger in the first reservoir is pressed, injecting the flushing solution into the vessel. Blood is flushed or displaced from the injection site. The second plunger is then pressed to inject the sclerosing agent into the vessel. By flushing the blood from the vessel, prior to injecting the sclerosing solution, the potential for blood leakage is greatly reduced. In addition, flushing the vessel with saline reduces the need to inject excessive amounts of sclerosing solution, thereby minimizing the potential of the mat-like telangiectasia complication.

In a fourth aspect of the invention, the potential for inadvertently causing skin ulcerations by injecting a sclerosing solution at an improper location is reduced or eliminated. A sterile saline or other flushing solution is injected first into the injection site. The physician visually observes the injection site. If the vein disappears or tends to fade from view, the needle is properly positioned in the vein, and has flushed out the blood. The sclerosing solution is then injected into the same site via the same needle, without withdrawing the needle from the site. On the other hand, if the vein appearance remains largely unchanged after the flushing solution is injected, the physician then has a visual indication that the needle is not properly positioned in the vein. The needle is then withdrawn and relocated, and the visual observation procedure is repeated. This method avoids inadvertent injection of sclerosing solution into tissue outside of the vein, and the potential complications, such as skin ulcerations, which may accompany such events.

In a fifth and separate aspect, a novel syringe assembly has two separate reservoirs connecting to a single needle. The liquid contents of the two reservoirs are separated from each during storage. Consequently, the syringe can be advantageously pre-filled with two different injectant solutions, and then optionally sealed in a package until use. The liquid contents are contained or sealed within the reservoirs by a plunger seal on a plunger towards the back end of the syringe assembly, and by an end cap or closed off needle at the front end. In use, an end cap is removed from the syringe assembly and a needle is attached, with the bore of the needle connecting into both reservoirs. Alternatively, a needle may be attached to the syringe assembly during or after manufacture, and no end cap is used. A needle tip protector/seal may optionally be pushed on to the tip of the needle, to avoid piercing packaging and needle stick incidents, and to prevent leakage from the reservoirs. The syringe assembly provides for improved sclerotherapy procedures, and may also be used for other procedures as well.

Other and further objects and advantages will appear. The invention resides as well in subcombinations of the methods and devices shown and described.

### BRIEF DESCRIPTION OF THE DRAWINGS

FIG. 1 is a perspective view of a syringe assembly for use in sclerotherapy, with a needle attached to the syringe assembly.

FIG. 2 is a section view taken along line 2-2 of FIG. 1.

FIG. 3 is enlarged perspective view of the end section of an alternative syringe design.

FIG. 4 is a perspective view of the syringe assembly of FIG. 1 with an end plug attached to the syringe assembly.

FIG. 5 is an enlarged perspective view of the end tube and end cap shown in FIG. 4.

FIG. 6 is an enlarged perspective view of the cap shown in FIGS. 4 and 5.

FIG. 7 is a side view, in part section, of an end cap on the end tube.

FIG. 8 is a schematic view of the needle shown in FIG. 1 or 2 inserted into a vessel or vein.

FIG. 9 is an enlarged partial section view of an alternative syringe assembly similar to the syringe assembly shown in FIG. 7, and having an end tube divider plate extending from the reservoir outlets to the front end of the syringe assembly.

FIG. 10 is a perspective view of an alternative end cap for use with the syringe assembly shown in FIG. 9.

### DETAILED DESCRIPTION

The invention provides sclerotherapy methods having reduced risk of complications. The methods involve flushing or displacing blood from the vein or vessel, before injecting a sclerosing solution into the vessel. Injection of a clear flushing solution, such as sterile saline displaces blood from the vein. The vein can then be difficult or impossible for the physician to see. Consequently, injecting the sclerosing solution through the same needle, at the same injection site, avoids the need to find the vein, after the flushing solution is injected. Consequently, the methods are more advantageously performed using a syringe assembly which can deliver both solutions with a single injection, through a single needle. This reduces the number of injections required. In addition, it ensures that the flushing and sclerosing solutions are injected at the same location, while avoiding the difficulties of finding the vein after injection of the flushing solution.

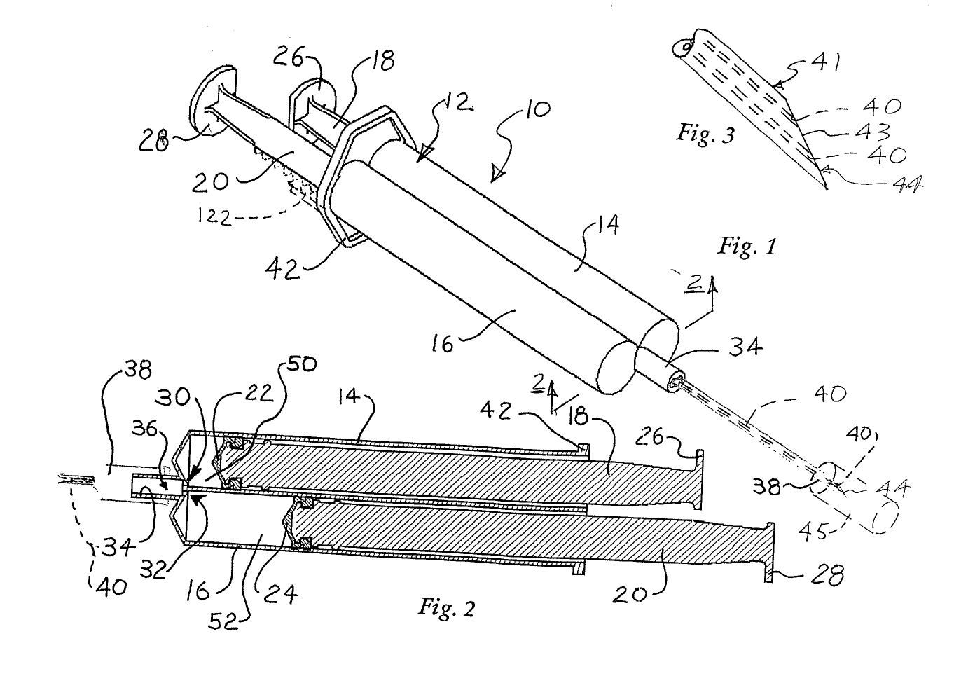

FIGS. 1 and 2 show a preferred syringe assembly for performing the methods described. As shown in FIG. 1, the syringe assembly 10 has a body 12, preferably formed as a single molded plastic unit. The body 12 includes first and second barrels, chambers or reservoirs 14 and 16. A first plunger 18 having a first end seal 22 is slidably positioned within the first reservoir 14. Similarly, a second plunger 20 having a second end seal 24 is slidably positioned within the second reservoir 16.

The first plunger 18 has a first cap 26 and the second plunger 20 has a second cap 28. The caps 26 and 28 are D-shaped, so that they can pass by each other. In use, the positions of the end caps 26 and 28 also provide a convenient visual and tactile indication of the relative volumes of fluids injected. For example, when they are aligned, the user knows that equal volumes have been injected. A hollow end tube 34 extends from the bottom or front end of the body 12. A bore or opening 36 in the end tube 34 connects into the first reservoir 14 through a first outlet 30. Similarly, the bore 36 in the end tube 34 also connects to the second reservoir 16 through a second outlet 32. The outlets 30 and 32 connect the reservoirs 14 and 16 directly into the bore 36. No valves or other flow control features are needed or used in this embodiment.

A hypodermic needle 38 having a needle bore 40 is attachable to the end tube 34 using e.g., a Luer fitting. Of course, other types of needles and fittings, bayonet, screw threads, etc., may also be used. A finger flange 42 is advantageously provided at the back or top end of the body 12. The first reservoir 14 is preferably filled with a flushing solution 50, preferably sterile saline solution. The second reservoir 16 is preferably filled with a sclerosing solution 52. The sclerosing agent may be sodium morruhate, sodium tetradecylsulfate, polilocanol, chromated glycerine, polyiodine iodine, hypotonic saline, Lauromacchogal, Abtysisclerol or other known sclerosing agent, in solution.

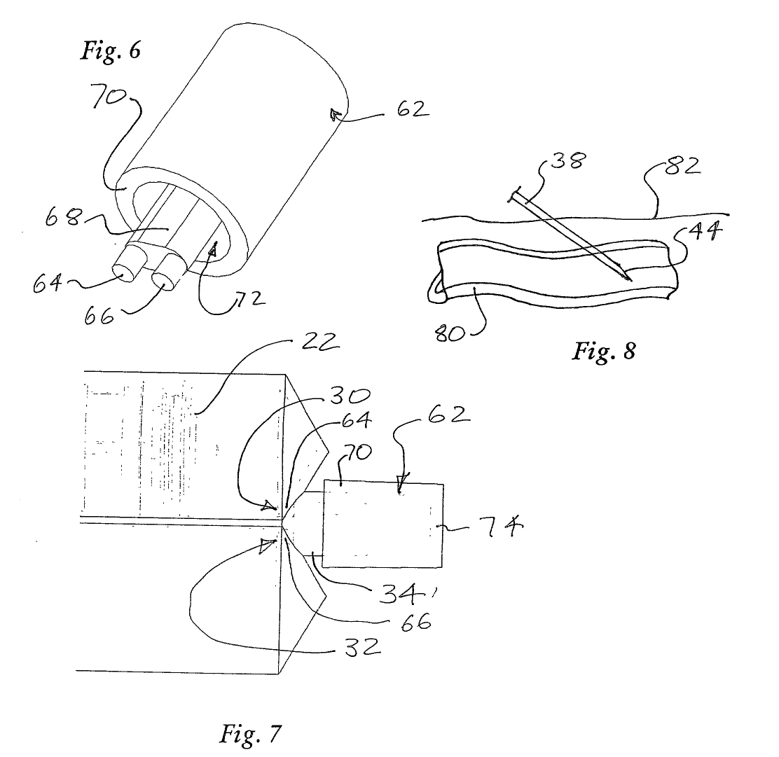

The syringe assembly 10 may advantageously be pre-filled with the solutions 50 and 52, with an end cap 62 on the end tube 34, to prevent leakage during shipment and storage, and to maintain sterility. An over package, envelope, or container 90, may optionally be provided, enclosing the syringe assembly 10, to further maintain sterility of the reservoir contents. With the reservoirs 14 and 16 filled, the plungers 18 and 20 are fully withdrawn. FIGS. 1 and 2 show the plungers at intermediate positions, for purposes of illustration. As shown in FIGS. 5-7, the end cap 62 has plugs 64 and 66 on a neck 68. The cylindrical body 70 of the end cap 62 surrounds, and is spaced apart from the neck 68 via a gap 72. The neck is attached to the front end or surface 74 of the end cap 62. The end cap 62 makes a friction fit onto the end tube 34, with the end tube 32 sliding into the gap 72. The plugs 64 and 66 move into and plug the outlets 30 and 32. Consequently, with the end cap 62 in place, the contents of the reservoirs 14 and 16 are sealed from the environment, and from each other. Providing the syringe assembly 10 as a prefilled unit avoids the need for the physician to separately fill the reservoirs.

Referring to FIG. 2, although there is a direct connection between the first and second reservoirs 14 and 16 and the bore 36 in the end tube 34 via the first and second outlets 30 and 32, the outlets 30 and 32 are plugged by the plugs 64 and 66 during storage. Any mixing between the solutions 50 and 52 after the end cap 62 is removed and the needle 38 attached, is inconsequential due to the relatively small size of the outlets 30 and 32, the flow characteristics through the syringe assembly, and the short duration of use of the syringe assembly after the end cap is removed. As no valves or other flow control devices are needed in this embodiment, injection of the solutions 50 and 52 is quick and simple. The syringe assembly itself is also a simple and inexpensive design.

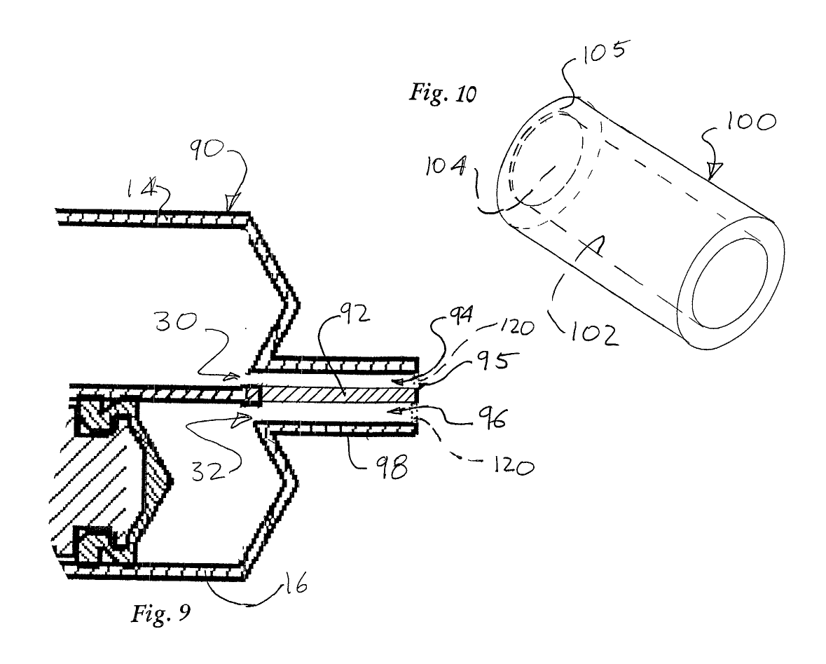

As shown in FIGS. 9 and 10, in an alternative syringe assembly 90, an end tube divider 92 extends from the outlets 30 and 32 to the very front end surface 95 of the end tube 98. This divides the end tube 98 into two separate bores 94 and 96. The end tube 98 preferably has a slightly tapering or conical outside wall. The end cap 100 shown in FIG. 10 has a complimentary inner wall 102. When the end cap 100 is pushed on to the end tube 98, it remains in place via the mating of the complimentary tapered surfaces and friction. The bottom end 105 of the end cap 100 contacts the front end surface 95 of the end tube 98 and seals off both of the separate bores 94 and 96. This prevents leaking or mixing of the contents of the reservoirs. A resilient end cap pad or disk 104 may optionally be attached to the bottom surface of the end cap, to help seal the ends of the bores 94 and 96 when the end cap 100 is installed. The plunger seals 22 and 24 seal off the back end of the reservoirs.

The syringe assembly may also be used to store and/or inject other combinations of solutions. In one such embodiment, the first reservoir contains a heparin solution and the second contains a saline solution.

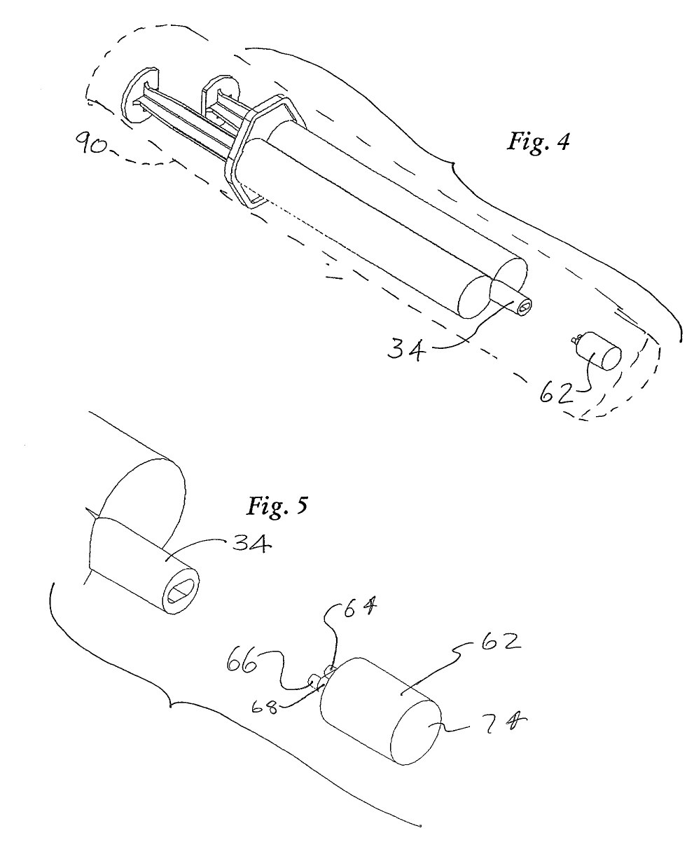

The needle 38 is preferably a 30-gauge needle. The needle preferably has a single lumen or bore 40 which connects to the outlets 30 and 32. This allows the single needle to deliver both solutions, while having a small diameter, and piercing only a small opening in the skin and vein. The needle 38 may be made part of, or be provided already attached to, the syringe assembly, with the syringe assembly/needle combination optionally provided with the package 90. As shown in dotted lines in FIG. 1, a needle cap 45 may be pushed or attached on to the tip of the needle. The needle cap helps to prevent piercing of the packaging, needle stick incidents, and leakage of the contents of the reservoirs out through the needle bore. The needle cap 45 may be resilient or rubber material. As the outlets 30 and 32 are both open into the bore of the needle, with this design, the contents of the reservoirs, over sufficient time, may diffuse into each other. If such diffusion must be entirely prevented, then the end cap 62 is installed in place of the needle, and the needle is installed only just before use. Alternatively, a needle 41 having two bores 40 may be used, as shown in FIG. 3, and with each bore separately connecting only to one of the reservoirs. With this design, separate flow paths from the outlets and through the tube 34 and needle 41 to the needle tip 43 are provided. Hence, in most ordinary uses any mixing of the solutions before injection is prevented, even with the needle attached to the syringe assembly during manufacture. However, the dual bore needle 41 necessarily requires a diameter larger than the single bore needle 38. The needle cap 45 can be used on needle 41 as well. The syringe assembly 10 shown in FIGS. 1-2 and 4-7 is used by removing the end cap 62 and attaching the needle 38 to the end tube 34. The design concepts described above may also be applied to a syringe assembly having three or more reservoirs.

Referring to FIGS. 2 and 9, as there is an open pathway between the outlets 30 and 32, either in the end tube 34, or within the bore of the needle, in rare situations, inadvertent mixing of the contents of the reservoirs may occur. Specifically, if flow through the needle bore is restricted, and the sliding friction of the plunger end seals 22 and 24 is sufficiently low, and if one of the plungers 18 or 20 is rapidly pushed in, then some liquid may flow from one reservoir into the other reservoir, causing inadvertent mixing. When this occurs, pushing on one plunger, e.g., 18, causes the other plunger 20 to move back out of the reservoir 16.

This result can be avoided in several ways. One way is to have the needle bore diameter (the I.D. of the needle) be larger than I.D. of the outlets 30 and 32. This will generally result in a flow resistance or pressure drop of flow through the needle bore, that is substantially less than flow resistance through either outlet 30 or 32. Hence liquid flowing out of outlet 30 will not flow into outlet 32. Typical hypodermic needles used with the syringe assemblies 10 or 90 range from Gauge 35 to Gauge 16. Gauge 35 has an O.D. of 0.012 and an I.D. of 0.006 inches. Gauge 16 has an O.D. of 0.065 and an I.D. of 0.053 inches. The bore diameters in this range are then nominally 0.006-0.013 inches. Accordingly, if the outlets are 0.005 inches or less, back flow mixing can be avoided in most or all cases. Outlet diameters of 0.001 -0.005 inches are preferred for this range of needles. While both outlets typically will have the same diameter, one may be larger, depending on the liquid content characteristics, purity requirements, etc.

Another alternative to prevent backflow mixing is use of one-way check valves or other flow restrictors 120, as shown in FIG. 9. The valves 120 prevent inflow into the reservoirs through the outlets 30 or 32. However, use of valves 120 requires that the reservoirs be filled from the top or back end, by removing the plungers, filling, and then replacing the plungers. A flow restrictor further increases flow resistance as a function of flow velocity, effectively limiting the speed of plunger movement and preventing back flow mixing.

Another alternative to prevent backflow mixing is a one-way ratchet or brake 122 as shown in FIG. 1. The ratchet 122, when engaged, prevents backward movement of the plunger. The ratchet may be disengaged temporarily, to fill the reservoir by pulling the plunger back up through the reservoir.

In use for sclerotherapy, needle 38 is oriented with the angled tip surface 44 up and is inserted into the vessel or vein 80 to be treated. Once inserted, the angle or bevel surface 44 of the needle is facing up towards the skin 82, as shown in FIG. 8. The first plunger 18 is pressed in, injecting the flushing solution into the vessel. The flushing solution displaces blood in the vessel, moving the blood away from the treatment site. Typically, the vein will no longer be visible once the blood is displaced. If the physician observes little or no change in appearance in the vein, then the needle is not properly located in the vein. The needle is then withdrawn and inserted at another location in the vein, before the sclerosing solution is injected. Injection of sclerosing solution outside of the vein can cause skin ulcerations. On the other hand, inadvertent injection of a flushing solution such as a saline outside of the vein creates no such risk of complications The use of flushing solution first, along with the change in appearance (or disappearance) of the vein, reduces potential complications resulting from injecting sclerosing solution outside of the vein.

After the flushing solution is injected, and the physician observes the change in appearance of the vein, and with the needle 38 remaining in place in the vessel, the second plunger 20 is then pressed in, injecting the sclerosing solution 52 into the vessel. As the sclerosing solution 52 acts on the vessel walls, without significant presence of blood at the treatment site, complications, such as hyperpigmentation, are reduced.

The volume of flushing solution 50 and sclerosing solution 52 used with each procedure may vary depending on the size of the vessel and other factors.

After the treating physician determines that a sufficient amount of solutions 50 and 52 have been injected, the needle 38 is withdrawn from the vessel. A pressure dressing may be applied to the skin around the injection site. The needle may be relocated to another treatment site, on the same vessel, or on a different vessel. The reservoirs hold 1-8, 2-6 or 3-5 ml each. These volumes allow for multiple injections with a single syringe assembly 10. Ultrasound treatment may be used on the areas treated over the 24-78 hour period following the above-described methods of sclerotherapy. This helps to further reduce or avoid bruising or potential hyperpigmentation.

The methods described above may also be used with electrosurgical techniques, such as described in U.S. Pat. Nos. 5,695,495 and 6,293,944, incorporated herein by reference, or with ultrasound image-guided techniques to locate the injection site, or both.

Thus, novel methods and devices have been shown and described. Various substitutions of steps and components may of course be made without departing from the spirit and scope of the invention. The invention, therefore, should not be limited, except by the following claims, and their equivalents.

1. A method for performing sclerotherapy on a human, comprising the steps of:
flushing blood out of a blood vessel of the human by injecting a sterile solution into the vessel, at an injection site; and
then injecting a sclerosing solution into the blood vessel at the injection site.

2. The method of claim 1 wherein the flushing step and the injecting step are performed by first supplying a flushing solution into the blood vessel through a needle inserted into the blood vessel, and then supplying the sclerosing solution into the blood vessel through the same needle, without removing the needle from the vessel.

3. A method for treating a spider vein or a varicose vein of a patient, comprising the steps of:
visually locating a treatment site on the vein by viewing the vein through the skin;
piercing a needle through the skin of the patient and into the vein;
flushing blood from a section of the vein, by injecting a sterile flushing solution into the vein, through the needle; and
injecting a sclerosing agent solution into the blood vessel, through the needle.

4. The method of claim 3 further comprising the step of leaving the needle in position in the vein until after the sclerosing agent solution is injected, so that the method is performed with only a single piercing of the skin.

5. The method of claim 3 wherein the flushing step is performed by supplying flushing solution from a first syringe to the needle, and the injecting step is performed by supplying the sclerosing agent solution from a second syringe attached to the first syringe, and also connecting to the needle.

6. A device for use in treatment of spider and varicose veins, comprising:
a first syringe containing a sterile solution;
a second syringe attached to the first syringe and containing a sclerosing agent;
a single hypodermic needle having a delivery port connecting to both the first syringe and the second syringe.

7. The device of claim 6 wherein the sterile solution comprises sterile saline solution.

8. The device of claim 6 wherein the sclerosing agent comprises a member selected from the group consisting of sodium morruhate, sodium tetradecylsulfate, polilocanol, chromated glycerine, hypotonic saline and polyiodine iodine.

9. The device of claim 6 wherein the first and second syringes are connected directly and continuously to the delivery port.

10. The device of claim 6 further comprising a first plunger in the first syringe, and a second plunger in the second syringe, with the first plunger movable independent of the second plunger.

11. The device of claim 6 further comprising an end cap closing off a first outlet into the first reservoir and closing off a second outlet into the second reservoir.

12. The device of claim 11 with the sterile solution sealed within the first reservoir by a first end seal on a first plunger and a first plug on the end cap, and with the sclerosing agent sealed within the second reservoir by a second end seal on a second plunger and a second plug on the end cap.

13. The device of claim 12 further comprising a sealed package enclosing the device.

14. A device for use in treatment of spider and varicose veins, comprising:
a first reservoir containing a sterile solution;
a second reservoir containing a sclerosing agent; and
a single hypodermic needle having a first bore and a second bore connecting respectively to the first reservoir and the second reservoir.

15. The device of claim 14 with the second reservoir attached to the first reservoir.

16. The device of claim 14 with the first reservoir sealed off from the second reservoir.

17. A syringe assembly comprising:
a first reservoir having a first outlet connecting into an end tube;
a second reservoir having a second outlet connecting into the end tube; and
an end cap on the end tube separating the first outlet from the second outlet.

18. The syringe assembly of claim 17 further comprising first and second sealing elements on the end cap engageable into the first and second outlets to seal the first reservoir and the second reservoir.

19. The syringe assembly of claim 17 with the end cap removable from the end tube, and further comprising a needle attachable to the end tube.

20. The syringe assembly of claim 19 with the first reservoir containing a heparin solution.
